(19) United States
(12) Patent Application Publication
(10) Pub. No.: US 2026/0007545 A1
(43) Pub. Date: Jan. 8, 2026

(54) OCULAR IMPLANT, KIT, METHOD OF DEPLOYING

(71) Applicant: Oxford University Innovation Limited, Oxford (GB)

(72) Inventors: Yunfang YANG, Oxford (Oxfordshire) (GB); Yunlan ZHANG, Oxford (Oxfordshire) (GB); Jared CHING, Oxford (Oxfordshire) (GB); Zhong YOU, Oxford (Oxfordshire) (GB)

(21) Appl. No.: 18/881,748

(22) PCT Filed: Jul. 10, 2023
(86) PCT No.: PCT/GB2023/051811

§ 371 Date: Jan. 7, 2025

(30) Foreign Application Priority Data
Jul. 12, 2022 (GB) 2210216.4

(51) Int. Cl. A61F 9/007

(52) U.S. Cl. CPC A61F 9/00781; A61F 2210/0076; A61F 2230/0017; A61F 2230/0069; A61F 2250/0067

(57) ABSTRACT

Ocular implants, kits and methods are disclosed. In one arrangement, an implant comprises a frame deployable in the suprachoroidal space of an eye and configured to resiliently adopt an arched elongate shape to promote drainage of aqueous humor through the suprachoroidal space when the frame is deployed in the suprachoroidal space. Relative to an axis of elongation of the elongate shape: the frame has a first axial end and a second axial end; and the arched elongate shape defines a channel. The channel has a first axial opening at the first axial end of the frame, a second axial opening at the second axial end of the frame, and a longitudinal opening extending continuously from the first axial end to the second axial end.

The present disclosure relates to ocular implants for treating glaucoma and associated kits and methods.

Glaucoma is the leading cause of irreversible blindness worldwide. In high pressure glaucoma, intraocular pressure reduction is known to prevent visual deterioration and loss.

The human eye comprises an anterior chamber and posterior chamber separated by the crystalline lens. The anterior chamber is filled with a plasma-like fluid called aqueous humor. When a person views an object, light reaches the retina after traversing various transparent structures of the eye, including the cornea, aqueous humor, crystalline lens and vitreous humor. A continuous flow of aqueous humor provides nutrition and removes wastes from these tissues, without the need for opaque vasculature that would impede light transmission. Aqueous humor is generated by the ciliary body behind the iris. It flows past the lens and iris and subsequently exits the anterior chamber through a number of drainage routes. The majority of the aqueous humor drains via the trabecular meshwork into Schlemm's canal. A small percentage exits via the uveoscleral drainage where aqueous humor enters the supraciliary muscle and drains through to the supraciliary space, before exiting via trascleral flow through emissary channels or by entering the choroidal vasculature.

The normal intraocular pressure (IOP) is 10-24 mmHg. When natural drainage routes do not function properly, a build-up of excess fluid in the eye causes a rise in IOP to a value consistently greater than normal limits. Over time, the high pressure causes damage to the optic nerve and results in loss of peripheral vision. As glaucoma progresses, gradual loss of visual field occurs until the patient is completely blind.

Treatments for glaucoma include medications, laser surgery, surgical procedures and minimally invasive implants.

Medications can include eye drops that control aqueous production and/or fluid inflow, or that facilitate outflow through the trabecular meshwork. These approaches may not be long lasting or complication-free, and poor patient compliance can lead to further symptom progression.

Surgery may include trabeculectomy. During this surgery, a small opening is created in the trabecular meshwork to allow the fluid to flow out of anterior chamber and a bleb is formed to store a small volume of subconjunctival aqueous humor temporarily, which is subsequently adsorbed by the posterior parts of the eye.

Minimally invasive implants may aim to avoid the potential complications and invasiveness of trabeculectomy. Such implants may be inserted into the eye to create an artificial drainage pathway or expand a naturally occurring drainage pathway.

US 2012/0123315 A1 discloses creation of drainage path by placing a tube structure between the anterior chamber and the sub-conjunctival space. U.S. Pat. No. 7,740,604 B2 discloses an implant that is inserted into Schlemm's canal. Other implants have targeted uveoscleral drainage via the suprachoroidal space. The suprachoroidal space can be accessed relatively easily via an ab interno approach and produces a significant IOP reduction. U.S. Pat. No. 9,788,999 B2 discloses a tubular implant that accesses through this route but was found to cause significant endothelial cell loss. US 2019/0038462 A1 discloses an implant intended to reduce endothelial cell loss by using a special biomaterial.

A further shortcoming found in some implants has been a vulnerability to occlusion caused by fibrosis of lumens.

It is an object of the present disclosure to improve treatment of glaucoma using ocular implants.

According to an aspect of the invention, there is provided an ocular implant comprising: a frame deployable in the suprachoroidal space of an eye and configured to resiliently adopt an arched elongate shape to promote drainage of aqueous humor through the suprachoroidal space when the frame is deployed in the suprachoroidal space, wherein relative to an axis of elongation of the elongate shape: the frame has a first axial end and a second axial end; and the arched elongate shape defines a channel having: a first axial opening at the first axial end of the frame; a second axial opening at the second axial end of the frame; and a longitudinal opening extending continuously from the first axial end to the second axial end.

The provision of an implant having a frame defining an arched elongate shape in the manner described above promotes high positional stability when deployed in the suprachoroidal space. Edge profiles of the frame that define opposite sides of the longitudinal opening are pressed into tissue to provide effective anchoring against longitudinal movement of the frame. The positional stability enhances longevity and reliability, as well as reducing or avoiding the need for the implant to protrude out of the suprachoroidal space, which reduces or avoids a risk of endothelial cell loss caused by the implant.

In some embodiments, one or each of the edge profiles extends non-linearly to increase friction and thereby inhibit longitudinal movement of the frame when deployed in the suprachoroidal space. Providing such non-linear edge profiles further enhances positional stability of the implant after deployment.

In some embodiments, the frame comprises a network of interconnected arms defining a plurality of cells defining respective openings in the frame. When deployed, the frame presses against tissue and causes the tissue to protrude into the openings of the cells. This protrusion of tissue into the openings anchors the frame with respect to longitudinal motion of the frame, thereby further improving positional stability.

In some embodiments, the channel has a cross-sectional area that is non-uniform along the axis of elongation. For example, the cross-sectional area may be smaller in a range of positions between the first and second axial ends than at one or both of the first and second axial ends. Varying the cross-sectional area has been found to allow the implant to conform with the shape of the suprachoroidal space and further enhance positional stability and reliable deployment.

Embodiments of the disclosure will now be further described, merely by way of example, with reference to the accompanying drawings.

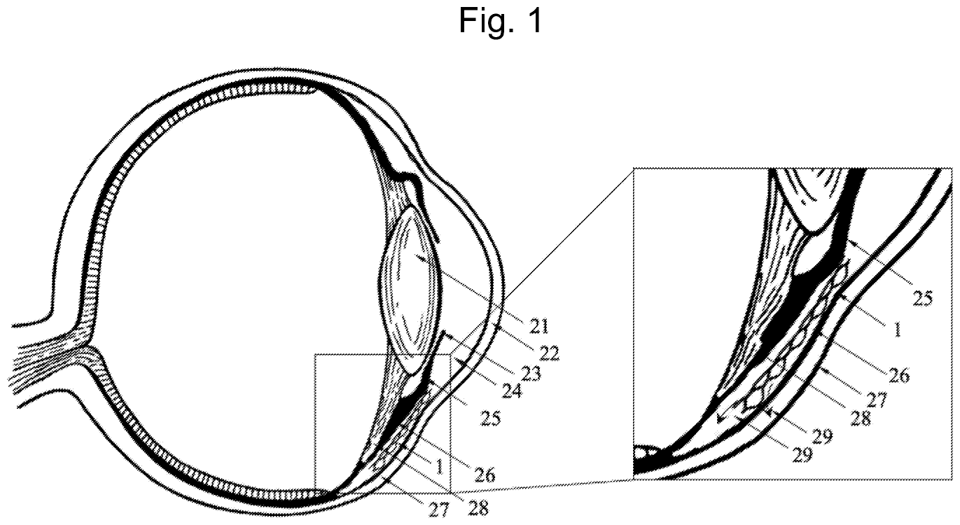

FIG. 1 is a plan view of a portion of an eye.

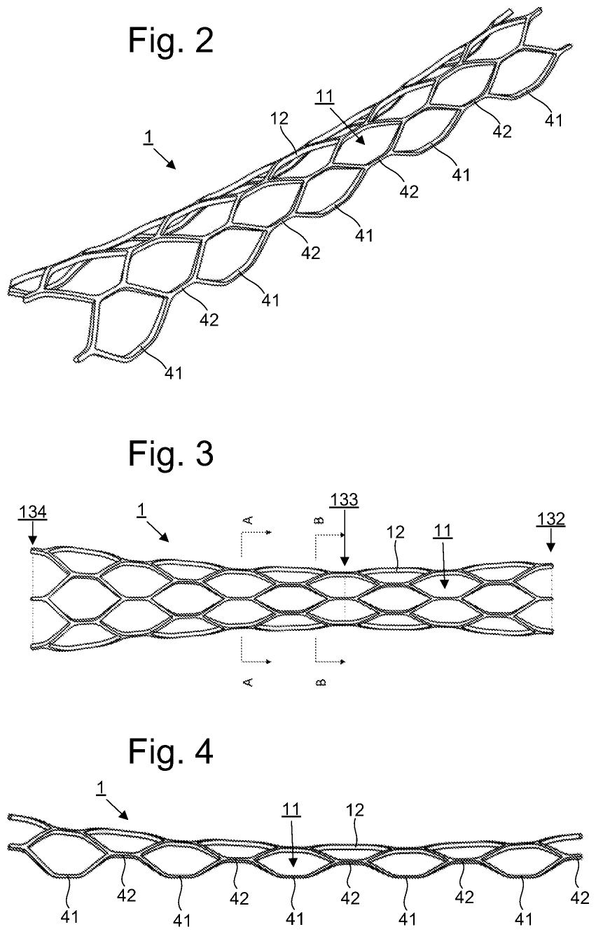

FIG. 2 is a perspective view of a frame of an implant.

FIG. 3 is top view of the frame of FIG. 2.

FIG. 4 is side view of the frame of FIG. 2.

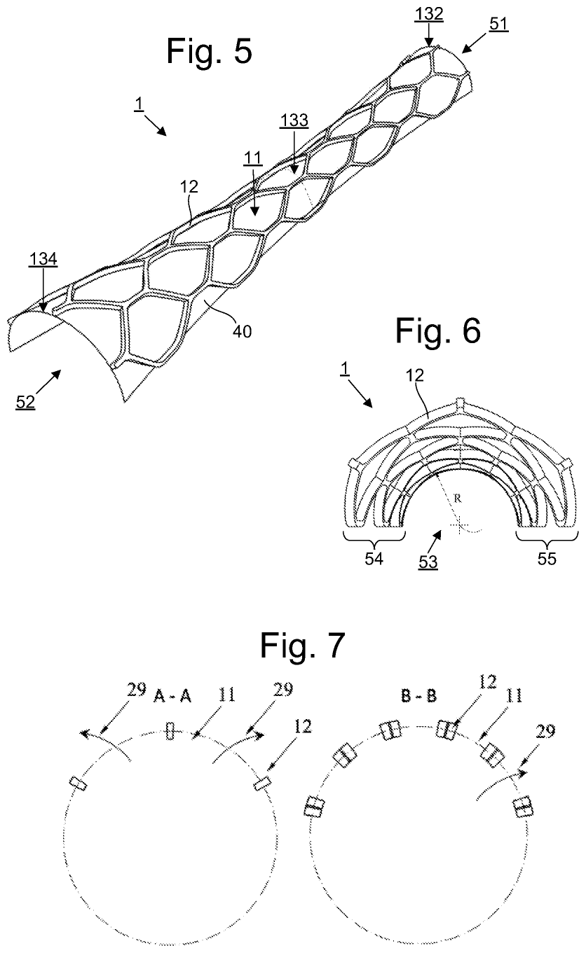

FIG. 5 is a perspective view of the frame of FIG. 2 with a notional reference surface shown to illustrate a geometry followed being radially inward facing surfaces of the frame.

FIG. 6 is an end view of the frame of FIG. 2 (viewed from the left relative to the orientation of FIG. 4).

FIG. 7 shows lateral cross-sectional views relative to planes A-A (left) and B-B (right) shown in FIG. 3.

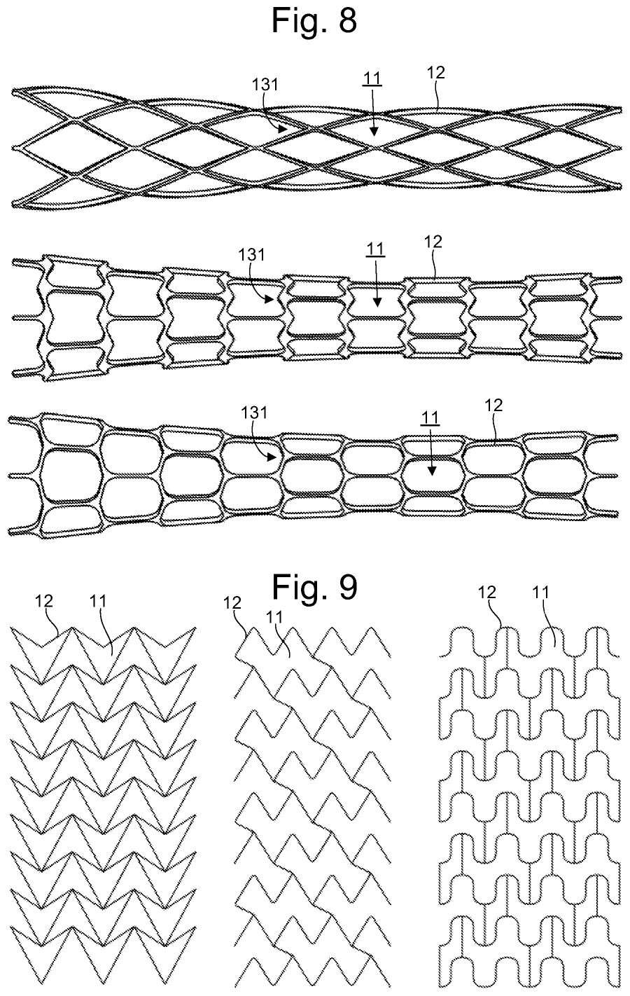

FIG. 8 depicts top views of three variations on the frame of FIG. 2.

FIG. 9 depicts top views of alternative frame tessellation patterns.

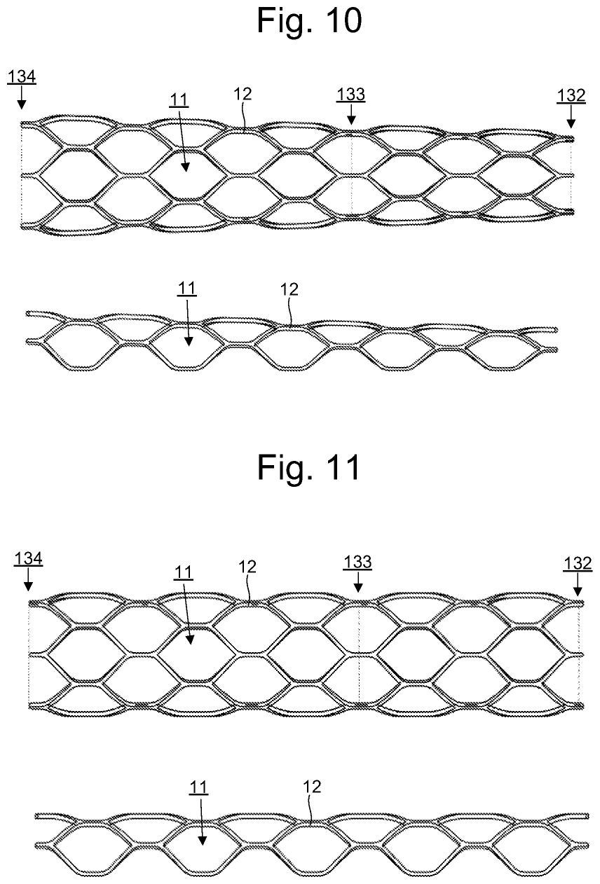

FIG. 10 depicts top (upper sub-figure) and side (lower sub-figure) views of a variation of the frame of FIG. 2 in which a cross-sectional area of a channel defined by the frame decreases monotonically from one longitudinal end to the other longitudinal end.

FIG. 11 depicts top (upper sub-figure) and side (lower sub-figure) views of a variation of the frame of FIG. 2 in which a cross-sectional area of the channel is substantially constant between the longitudinal ends.

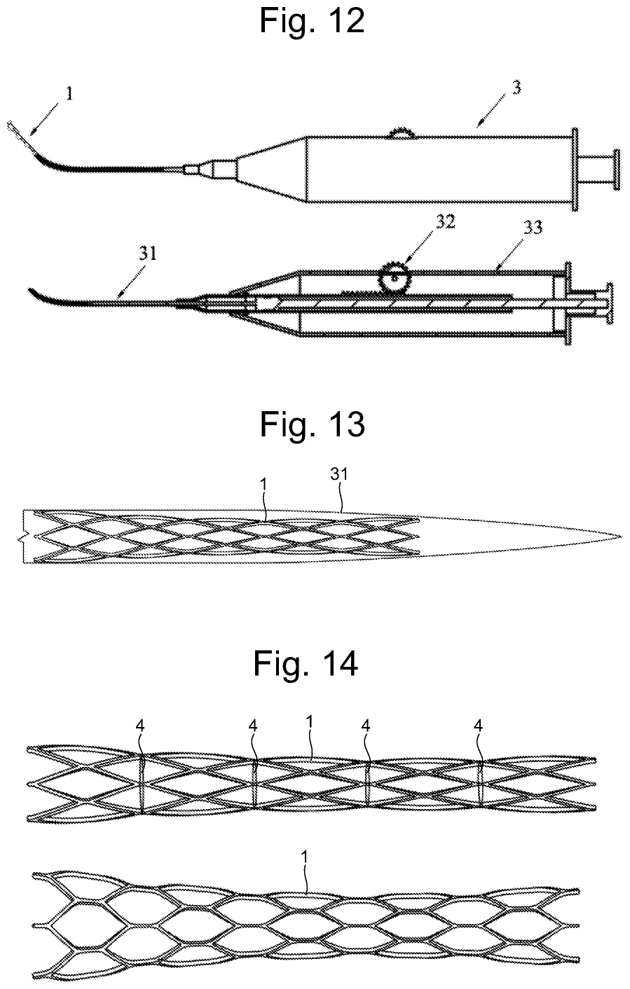

FIG. 12 depicts side (upper sub-figure) and side sectional (lower sub-figure) views of a delivery system.

FIG. 13 is a plan view of a frame in a delivery sheath, the frame constrained in a radially contracted state by the delivery sheath.

FIG. 14 shows a top view (upper sub-figure) of a frame constrained in the radially contracted state by a constraining material and the same frame (lower sub-figure) after the constraining material has broken down in the suprachoroidal space to release the frame.

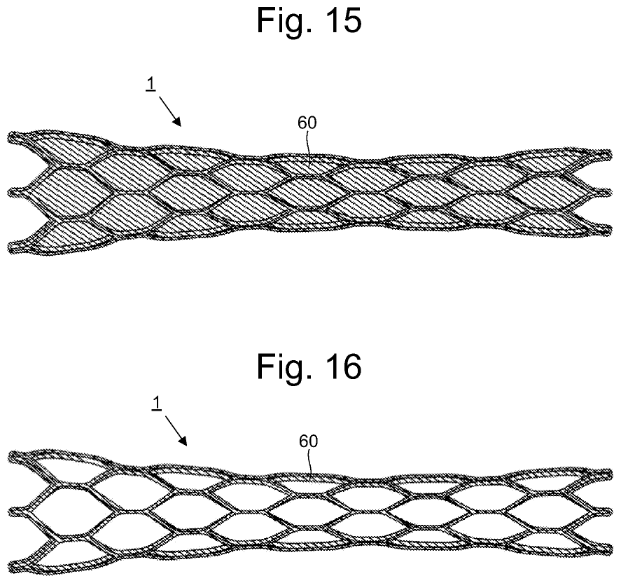

FIG. 15 depicts a variation on the arrangement of FIG. 3 in which a coating in the form of a membrane is provided on the frame.

FIG. 16 depicts a variation on the arrangement of FIG. 3 in which a coating is provided on arms of the frame without spanning across openings of the frame.

FIG. 1 is a plan view of a portion of an eye. The crystalline lens 21 and iris 23 of the eye are visible through cornea 22. Cornea 22 encloses an anterior chamber 24, which is filled with aqueous humor that inflates the globe of the eye and maintains the intraocular pressure. Aqueous humor is secreted from the ciliary body 28, which also supports the lens 21. The aqueous humor is continually produced and flows through the eye to provide nutrition, removes the waste from tissues and maintains the hemi-spherical shape of the cornea. As mentioned in the introductory part of the description, the aqueous humor can drain via several routes. A first route is through the trabecular meshwork 25 into Schlemm's canal, which is where most of the aqueous outflow occurs. The second route is uveoscleral drainage, in which aqueous flow passes between the muscle between the anterior chamber and the ciliary muscle into the supraciliary and suprachoroidal space 26, before being drained through the sclera 27.

Embodiments of the present disclosure provide an ocular implant configured for deployment in the suprachoroidal space 26. The implant may be inserted into this position via the anterior chamber 24. When in place, the implant promotes flow of aqueous humor in the suprachoroidal space (thereby facilitating uveoscleral drainage) by supporting and/or expanding the suprachoroidal space.

An example embodiment is depicted in FIGS. 2-7. The implant comprises a frame 1. The frame 1 is deployable in the suprachoroidal space of an eye. The frame 1 may thus be formed of a biocompatible material and be shaped and dimensioned to allow safe deployment as an implant in this particular location. For example, the frame 1 may have a length in the range of about 3 mm to about 8 mm. A width of the frame 1 may be in the range of about 0.2 mm to about 3.5 mm, optionally in the range of about 0.4 mm to about 1.5 mm. The dimensions of the frame 1 may be custom made and/or selected to correspond to the particular anatomy of a patient's eye.

The frame 1 is configured to resiliently adopt an arched elongate shape to promote drainage of aqueous humor through the suprachoroidal space when the frame 1 is deployed in the suprachoroidal space. The frame 1 may thus press radially outwardly against tissue in the suprachoroidal space. The elongate shape has an arched cross-section perpendicular to an axis of elongation of the elongate shape. The axis of elongation runs from the bottom-left to the top-right in FIGS. 2 and 5, from left to right in FIGS. 3 and 4, and perpendicularly into the page in FIGS. 6 and 7. The cross-section is typically arched along the whole length of the elongate shape. The cross-section may be constant along the length or may vary (as exemplified and discussed in further detail below; see FIG. 6 for example).

Relative to the axis of elongation, the frame 1 has a first axial end 132 and a second axial end 134. The elongate shape defines a channel for promoting drainage of aqueous humor through the suprachoroidal space 26. The channel has a first axial opening 51 at the first axial end 132 of the frame 1. The channel has a second axial opening 52 at the second axial end 134 of the frame 1. The channel has a longitudinal opening 53 (see FIG. 6) extending continuously from the first axial end 132 to the second axial end 134 (i.e., underneath the frame 1 in the orientation of the figures).

The elongate shape is elongate in the sense that a length of the shape along the axis of elongation is greater than a largest linear dimension of any cross-section of the channel, for example greater than a diameter of the channel at all positions between the first and second axial ends of the frame 1. In the orientation of FIGS. 3 and 4, for example, the length of the shape corresponds to the distance from the leftmost side of the frame 1 to the rightmost side of the frame 1. A largest linear dimension of any cross-section of the channel corresponds to the broken line depicted at the second axial end 134 in FIG. 3, which is the diameter of the channel at the second axial end 134.

FIG. 2 is a perspective view of the frame 1 in the deployed state. FIG. 5 is a version of FIG. 2 with a notional reference surface 40 shown to illustrate a geometry followed by radially inward facing surfaces of the frame 1. The reference surface 40 is not physically part of the frame 1 but merely facilitates visualisation of the arched elongate shape adopted by the frame 1. The frame 1 is configured such that radially inward facing surfaces of the frame 1 follow the geometry of the reference surface 40 (e.g., if the reference surface 40 were a real surface, the radially inward facing surfaces of the frame 1 would be flush against the reference surface 40). The frame 1 may be formed by laser cutting a tube and shape set into particular shape to accommodate the anatomy of suprachoroidal space, in which case the reference surface 40 would correspond to the shape of the shape setting tool inside the frame 1.

In some embodiments, as exemplified in FIG. 5, the arched elongate shape adopted by the frame 1 may have a cross-section resembling a portion of a circle. In such embodiments, the portion of the circle may subtend an angle between 90 degrees and 270 degrees, preferably between 120 degrees and 240 degrees, preferably between 150 degrees and 210 degrees, preferably substantially 180 degrees, for at least a portion of a length of the frame 1. In the example of FIGS. 2-7, the arched elongate shape has a cross-section resembling a semicircle (which subtends substantially 180 degrees).

The frame 1 may be configured to self-expand from a radially contracted state to a radially expanded state. The arched elongate shape may correspond to the radially expanded state. The frame 1 can be inserted into a desired position in the eye (e.g., in the suprachoroidal space) while being held in the radially contracted state and then released to spring out into the radially expanded state. The self-expanding property may be provided, for example, by forming the frame from an elastic material or a shape-memory material such as nitinol or similar. Further examples of materials for the frame 1 are given below.

In some embodiments, the frame 1 comprises edge profiles 54, 55 (labelled in FIG. 6 for example) defining opposite sides of the longitudinal opening 53. The edge profiles 54, 55 engage against tissue in use. In the orientations of FIG. 2-7, the edge profiles are defined by the lowest extremities of the frame 1 on either side of the longitudinal opening 53. The resilient nature of the frame 1 means that the frame 1 pushes radially outwards against tissue when deployed. In the orientation shown in FIG. 2-7, the bulbous upper portion of the frame pushes upwards against tissue and the edge profiles 54, 55 push downwards. Due to the relatively small surface area of the edge profiles 54, 55, the edge profiles 54, 55 embed into tissue and provide an anchoring force against longitudinal movement of the frame 1, thereby enhanced positional stability of the implant.

In some embodiments, the edge profiles extend non-linearly along the length of the frame 1. The non-linear form increases friction and thereby further inhibits longitudinal movement of the frame 1 when deployed in the suprachoroidal space. The non-linear edge profiles 54, 55 in such embodiments may be referred to as anchoring structures. In the example of FIGS. 2-7, both edge profiles 54, 55 are non-linear. The edge profiles undulate along the length of the frame 1 (e.g., left to right in FIG. 4) defining protrusions 41 and recesses 42. The edge profiles 54, 55 consist of the protrusions 41 and recesses 42 in this example. When deployed in the suprachoroidal space, the protrusions 41 will press into tissue and tissue will extend into the recesses 42. Both effects will act to inhibit unwanted longitudinal movement of the frame 1 after deployment.

In some embodiments the frame 1 comprises a network of interconnected arms 12. The arms 12 may be referred to as struts. Where the arms 12 are formed from metal materials (e.g., nitinol), a thickness of the arms (struts) may typically be in the range of about 0.03 mm to 0.05 mm for example. Where the arms 12 are formed from biocompatible polymer materials, the thickness of the arms (struts) may typically be in the range of about 0.03 mm to 0.09 mm, depending on the flexibility of the material. The network may be formed by laser cutting a tube (e.g., a tube having a semi-circular cross-section or a cylindrical tube that is subsequently cut to form the arched elongate shape) or by any other suitable method. The arms 12 define closed-loop cells 11. Each cell 11 defines an opening (e.g., a radially facing opening) in the frame 1 surrounded by one or more of the arms 12. In the example of FIGS. 2-7, each cell 11 is surrounded by six substantially linear arms 12 that together form a hexagon. The opening of each cell 11 is thus hexagonal in this example. The network of arms 12 forms the arched elongate shape that defines the channel. In the orientation shown in FIGS. 2-7, the openings cause the bulbous upper portion of the frame 1 to be porous. Material coverage in this porous bulbous upper portion will typically be less than 50% but is not particularly limited.

When deployed in the radially expanded state in the suprachoroidal space, the frame 1 presses against tissue. In some embodiments, this causes tissue to protrude into the openings of the cells 11. Protrusion of tissue into the openings of the cells 11 anchors the frame 1 in position with respect to longitudinal motion of the frame 1. The protrusion of tissue thus inhibits unwanted longitudinal movement of the frame 1. In other embodiments, for example where a coating in the form of a membrane is provided that covers the openings 11, the tissue may not protrude significantly or at all into the openings of the cells 11. An example frame 1 having such a coating is shown in FIG. 15.

In some embodiments, the cells 11 are provided in rows aligned perpendicularly with respect to the axis of elongation of the elongate shape (e.g., along a circumferential direction). The number of cells 11 in the rows may alternate along the axis of elongation for at least a portion of the frame 1. In the example of FIGS. 2-7, the number of cells 11 in the rows alternates between 2 and 3. The number of cells 11 in each row is typically smaller than the number of rows due to the elongate nature of the frame 1.

In the embodiment shown, the cells 11 are hexagonal and tessellate in a honeycomb configuration. In other embodiments, as exemplified in FIG. 8, the cells may have other shapes. Different patterns of cells 11 can be obtained in various ways during a design process. In the example of FIG. 8, different patterns are obtained by varying on interior angle 131 of the cells 11 and/or selectively removing arms 12. For example, when the angle 131 is less than 120 degrees and a longitudinally aligned arm 12 is removed, the cells 11 can form a tessellating pattern of rhombi as exemplified in the upper sub-figure of FIG. 8. When the angle 131 is made larger than 180 degrees, the cells 11 can form inverted hexagonal polygons as exemplified in the middle sub-figure of FIG. 8). When the angle 131 is set between 120 and 180 degrees, the cells 11 can be made approximately circular as exemplified in the lower sub-figure of FIG. 8. FIG. 9 shows further possible patterns for the network of arms 12 that may allow more compact compression of the frame 1 in the pre-deployed (radially contracted) state. The thicknesses and/or compositions of the arms 12 can be tuned during the design process to select a desired stiffness for the frame 1.

The dimensions and/or stiffness of the elongate arch defined by the frame 1 are selected to support and slightly expand the tissue in suprachoroidal space. In some embodiments, the frame 1 is configured such that, when viewed along the axis of elongation, cross-sections along the length of the frame 1 each follow a portion of a circular path having a radius in the range of 0.15-1 mm.

In some embodiments, the frame 1 is configured such that the channel has a cross-sectional area that is non-uniform along the axis of elongation. The cross-sectional area thus varies along the longitudinal direction, optionally by at least 5%, optionally at least 10%, optionally at least 20%, optionally at least 40%. Providing such a variation may enhance longitudinal anchoring of the frame 1 after deployment (inhibiting longitudinal movement of the frame 1). In some embodiments, as exemplified in FIGS. 2-7, the cross-sectional area is smaller in a range of positions between the first and second axial ends 132, 134 than at one or both of the first and second axial ends area 132, 134, optionally smaller by at least 5%, optionally at least 10%, optionally at least 20%, optionally at least 40%. The variation may cause the frame 1 to adopt a saddle shape. Such arrangement may provide effective longitudinal anchoring against movement in both longitudinal directions. The variation in area is highlighted by the reference surface 40 in FIG. 5 for the embodiment of FIGS. 2-7. The radius of the channel is shown to vary continuously as a function of longitudinal position. The radius decreases from a maximum value at the second axial end 134 to a minimum value at intermediate position 133 before increasing again to a larger value at the first axial end 132. The radius at the second axial end 134 may be different to the radius at the first axial end 132. Such a variation in radius has been found to provide particularly effective embedding of the frame 1 into the sclera and, as a consequence, a high level of positional stability (e.g., resistance to displacements in either longitudinal direction). In some embodiments, the frame 1 is also curved in a saddle shape to better accommodate the round shape of the suprachoroidal space in the human eye.

In other embodiments, as exemplified in FIG. 10, the cross-sectional area decreases monotonically from one axial end 134 to the other axial end 132. The radius may thus decrease monotonically, for example linearly, from one axial end 134 to the other axial end 132. Interior surfaces of the frame 1 may, for example, conform to a portion of a cone. Embodiments having such a monotonic variation in area (and/or radius) may provide a larger anchoring force with respect to movement in one longitudinal direction than the other longitudinal direction. For example, when positioning in the eye, the frame 1 of FIG. 10 may be moved more easily to the left than to the right. The variation may also improve conformity between the shape of the frame 1 and the anatomical space in the frame 1 is to be deployed. Alternatively, as exemplified in FIG. 11, the cross-sectional area (and/or radius) may be made the same over the whole length of the frame 1, for example such that the frame 1 follows a cylindrical geometry. This approach may facilitate manufacture of the frame 1.

The flow of aqueous humor into and out of the channel will typically occur mainly via the first and second axial openings 51 and 52 of the channel. However, flow may also occur through openings in the side walls of the frame 1, such as the openings defined by the cells 11, as shown in FIG. 7 and indicated by arrows 29. Flow through the side walls may occur, for example, where a small portion of the frame 1 protrudes out of the suprachoroidal space into the anterior chamber.

The frame 1 can be fabricated from various biocompatible materials with dimensions selected to provide the desired structural and mechanical attributes. Metallic or non-metallic materials may be used. Examples of metallic materials include stainless steel, tantalum, titanium, nitinol (mentioned above), and cobalt-chromium. In some embodiments, the frame 1 may be provided with a biocompatible coating that improves anchorage performance, enhances biointegration, and/or provides antifibrotic properties. As described below with reference to FIGS. 15 and 16, the biocompatible coating may be provided as a membrane spanning the openings 11 or may be provided only on the frame 1, leaving the openings 11 open. Alternatively or additionally, the frame 1 may include a therapeutic agent supported by the frame 1. The therapeutic agent may be incorporated into a polymeric coating that is deposited on an outer and/or inner surface of the frame 1. In some embodiments, the therapeutic agent comprises an anti-glaucoma drug and/or a biodegradable drug matrix. Examples of anti-glaucoma drugs include prostaglandin analogues.

In an embodiment, a kit is provided for deploying the implant into the suprachoroidal space. The kit may comprise an implant according to any of the embodiments discussed herein. As exemplified in FIG. 12, the kit may further comprise a delivery system 3 configured to deliver the implant to the suprachoroidal space. The implant may be provided pre-installed or encapsulated in the delivery system 3. The delivery system 3 may comprise a delivery sheath 31 containing the implant 1. The delivery sheath 31 may be configured (e.g., shaped and/or dimensioned) to be inserted into the suprachoroidal space via the anterior chamber of the eye. The delivery system 3 may be configured such that the delivery sheath 31 can be withdrawn from the suprachoroidal space while leaving the implant within the suprachoroidal space.

An example of such a delivery system 3 is depicted in FIG. 12. The delivery system 3 of this embodiment comprises a handle 33 and a retrieval mechanism 32. The retrieval mechanism 32 comprises a gear and rack configured to drive relative movement between a delivery sheath 31 and an implant (comprising a frame 1) inside the delivery sheath 31. A core member comprising a wire or tube may be provided inside the delivery sheath 31 to allow the delivery sheath 31 to be withdrawn without a corresponding movement of the implant. In an embodiment, a lumen of the delivery sheath 31 is smaller than a radius of the implant when the frame 1 is in a radially expanded state such that the implant is constrained to be in a radially contracted state when in the delivery sheath 31. FIG. 13 is a top view showing the frame 1 of an implant in the radially contracted state within the delivery sheath 31. A size of the core member is defined by the size of the delivery sheath 31. After insertion into the anterior chamber, a distal end of the delivery sheath 31 is advanced into the suprachoroidal space until the frame 1 of the implant reaches a desired position. The retrieval mechanism 32 is then actuated to pull the delivery sheath 31 backwards relative to the frame 1 while the frame 1 is maintained at the same position by the core member, thereby deploying the frame 1. After the implant is fully deployed, the core member and sheath 31 may be withdrawn from the suprachoroidal space.

Alternatively or additionally, the frame 1 may be held in the radially contracted state by a constraining material that is configured to break down in the suprachoroidal space and release the frame 1 into the radially expanded state in a desired deployment position. In embodiments of this type, the frame 1 may be deployed without a delivery sheath 31 (i.e., bare). An example configuration of this type is depicted in FIG. 14 (upper sub-figure), where the constraining material forms localized loops 4. The constraining material may comprise a biocompatible substance such as a suture material (e.g., polyglycolic acid and/or polymers such as polyethylene glycol and/or polylactic-co-glycolic acid). After insertion, the constraining material 4 breaks down over time, for example by being hydrated by the aqueous humor and gradually dissolving. The constraining material 4 eventually releases the frame 1 to expand into the radially expanded state.

In some implementations, as exemplified in FIGS. 15 and 16, the implant may further comprise a biocompatible coating 60. The coating 60 is provided on the frame 1. The coating 60 may be provided on either or both of the radially inner and outer surfaces of the frame 1. The coating 60 may comprise silicone or any other biocompatible material. The coating 60 may reduce or prevent scarring around the frame 1 after deployment in the eye and/or otherwise improve biocompatibility. The coating 60 may be configured to enhance biointegration, for example by comprising a microporous structure and/or a multi-layered coating with a network of micropores. Alternatively or additionally, the coating may improve anchorage performance. Alternatively or additionally, the coating may provide antifibrotic properties. In some embodiments, the coating 60 may comprise a therapeutic agent such as an anti-glaucoma drug. The coating 60 may be configured to be drug-eluting.

FIG. 15 depicts a variation on the arrangement of FIG. 3 in which such a coating 60 is provided on a radially outer surface of the frame 1. The coating 60 is provided in the form of a membrane that spans across the openings 11, for example in the form of a thin web.

FIG. 16 depicts a variation on the arrangement of FIG. 3 in which such a coating 60 is provided on a radially outer surface of the frame 1 without spanning across the openings. The coating 60 in this case is provided exclusively in contact with the frame 1.

1. An ocular implant comprising:
a frame deployable in the suprachoroidal space of an eye and configured to resiliently adopt an arched elongate shape to promote drainage of aqueous humor through the suprachoroidal space when the frame is deployed in the suprachoroidal space, wherein relative to an axis of elongation of the elongate shape:
the frame has a first axial end and a second axial end; and
the arched elongate shape defines a channel having:
a first axial opening at the first axial end of the frame;
a second axial opening at the second axial end of the frame; and
a longitudinal opening extending continuously from the first axial end to the second axial end.

2. The implant of claim 1, wherein:
the frame comprises edge profiles defining opposite sides of the longitudinal opening, and one or each of the edge profiles extends non-linearly to increase friction and thereby inhibit longitudinal movement of the frame when deployed in the suprachoroidal space.

3. The implant of claim 2, wherein the non-linear edge profile comprises a plurality of protrusions and recesses.

4. The implant of claim 1, wherein the frame comprises a network of interconnected arms defining a plurality of cells defining respective openings in the frame.

5. The implant of claim 4, wherein the cells are provided in rows aligned perpendicularly with respect to the axis of elongation of the elongate shape.

6. The implant of claim 5, wherein the number of cells in the rows alternates along the axis of elongation.

7. The implant of claim 1, wherein the channel has a cross-sectional area that is non-uniform along the axis of elongation.

8. The implant of claim 7, wherein the cross-sectional area is smaller in a range of positions between the first and second axial ends than at one or both of the first and second axial ends.

9. The implant of claim 8, wherein the cross-sectional area is smaller in the range of positions than at both of the first and second axial ends.

10. The implant of claim 8, wherein the cross-sectional area decreases monotonically from one axial end to the other axial end.

11. The implant of claim 1, wherein the arched elongate shape is a radially expanded state and the elongate frame is configured to self-expand from a radially contracted state into the radially expanded state.

12. The implant of claim 11, wherein the frame is held in the radially contracted state by a constraining material that is configured to break down in the suprachoroidal space and release the frame into the radially expanded state.

13. The implant of claim 1, further comprising a biocompatible coating provided on or in the frame, the coating configured to improve anchorage performance, enhance biointegration and/or provide antifibrotic properties.

14. The implant of claim 1, further comprising a therapeutic agent supported by the frame.

15. A kit for deploying an implant into the suprachoroidal space of an eye, comprising:
the implant of claim 1; and
a delivery system configured to deliver the implant to the suprachoroidal space of an eye.

16. The kit of claim 15, wherein the delivery system comprises a delivery sheath containing the implant and configured to be inserted into the suprachoroidal space via the anterior chamber of the eye, the delivery system being configured such that the delivery sheath can be withdrawn from the suprachoroidal space while leaving the implant within the suprachoroidal space.

17. A method of deploying an implant into the suprachoroidal space, comprising deploying the implant of claim 1 into the suprachoroidal space.
